(19) United States
(12) Patent Application Publication
(10) Pub. No.: US 2022/0073969 A1
(43) Pub. Date: Mar. 10, 2022

(54) USE OF SIDEROMYCINS TO LIMIT CROSS-REACTIVITY AND IMPROVE SPECIES IDENTIFICATION IN ANTIBIOTIC SUSCEPTIBILITY ASSAYS

(71) Applicant: Roche Molecular Systems, Inc., Pleasanton, CA (US)

(72) Inventors: Kyle C. Cady, Santa Clara, CA (US); Ryan Chan, San Jose, CA (US)

(21) Appl. No.: 17/418,038

(22) PCT Filed: Dec. 22, 2019
(86) PCT No.: PCT/EP2019/086886

§ 371 Date: Jun. 24, 2021

(51) Int. Cl. C12Q 1/689; C12Q 1/6897

(52) U.S. Cl. CPC C12Q 1/689; C12Q 1/6897

(57) ABSTRACT

The present invention relates to the use of sideromycins as additives in non-replicative transduction particles based systems either to limit cross-reactivity of unwanted organisms or to identify the organism being run on an antibiotic susceptibility assay (AST assay). Addition of the sideromycins removes or reduces light production from bacteria that are sensitive to them, allowing for prevention of cross-reactivity in AST assays and/or family, genus, and potentially species level bacteria strain identification when performing AST testing.

### CROSS REFERENCE TO RELATED APPLICATIONS

This application is a U.S. national stage filing under 35 U.S.C. § 371 of International Application No. PCT/EP2019/086886, filed Dec. 22, 2019, entitled “USE OF SIDEROMYCINS TO LIMIT CROSS-REACTIVITY AND IMPROVE BACTERIAL IDENTIFICATION IN ANTIBIOTIC SUSCEPTIBILITY ASSAYS”, which claims the benefit of priority to U.S. Provisional Patent Application No. 62/786,431 filed on Dec. 29, 2018, and to U.S. Provisional Patent Application No. 62/858,146 filed on Jun. 6, 2019, each of which is hereby incorporated in its entirety by reference.

### BACKGROUND OF THE INVENTION

A transduction particle refers to a virus capable of delivering a non-viral nucleic acid into a cell. Viral-based reporter systems have been used to detect the presence of cells and rely on the lysogenic phase of the virus to allow expression of a reporter molecule from the cell. These viral-based reporter systems use replication-competent transduction particles that express reporter molecules and cause a target cell to emit a detectable signal.

Recently, methods and systems for packaging reporter nucleic acid molecules into non-replicative transduction particles (NRTPs), also referred herein as Smarticles, have been described in U.S. Pat. No. 9,388,453 and in U.S. Patent Application Publication No. 2017/0166907 (both of which are incorporated herein by reference in their entireties) in which the production of replication-competent native progeny virus nucleic acid molecules were greatly reduced due to the disruption of the packaging initiation site in the bacteriophage genome Cell-reporter systems can exhibit cross-reactivity and microbial interference with non-target organisms. For example, if an Enterobacteriaceae reporter is used to detect E. coli in a stool sample; other species of Enterobacteriaceae such as K. pneumoniae may produce a cross-reactive signal resulting in a false positive result. Furthermore, species of other Family of bacteria, such as P. aeruginosa, A. baumannii, and S. maltophilia, which may be present in a sample, may result in microbial interference resulting in a false negative result.

Antimicrobial susceptibility tests (AST) measure the response of a microorganism to an antimicrobial and are used to determine if the microorganism is susceptible or non-susceptible to the antimicrobial. The response of a microorganism to an antimicrobial may be due to a variety of mechanisms, all of which give the same response or phenotype. For example, in carbapenem resistant Enterobacteriaceae (CRE), resistance to carbapenem antibiotics may be due to a variety of carbapenemases encoded by different genes and gene variants including bla_{NDM-1}, bla_{KPC}, bla_{IMP}, bla_{VIM}, bla_{CMY}, etc. as well as situations that result in a carbapenem non-susceptible phenotype despite the lack of a carbapenemase such as non-carbapenemase β-lactamase hyper-expression and mutations that result in decreased uptake of a carbapenem into a cell (e.g. porin mutations).

Therefore, there is a need to limit or eliminate the problem of cross-reactivity of unwanted organism when performing AST assays with cell reporter systems (e.g. the Smarticles NRTP system). Bacteria and fungi have evolved highly specific iron sequestration processes that involve energy dependent active transport of relatively low molecular-weight iron chelators called siderophores (Raymond, K. N.; Dertz, E. A. “Biochemical and physical properties of siderophores.” In Iron Transport in Bacteria; Crosa, J. H., Mey, A. R., Payne, S. M., Eds.; American Society for Microbiology, 2004; pp 3-17). In Gram-negative bacteria, iron-siderophore complexes are preferentially recognized and bound by specific outer membrane receptors (OMR)/transporters. Binding of the siderophore-iron complexes initiates an energy-dependent active transport process that translocates the iron complex to the periplasm. This is often followed by active transport through the inner membrane. Hundreds of structurally distinct microbial siderophores have been identified, and more are discovered and reported frequently. The structural diversity is not a biosynthetic waste or redundant but careful evolution based on combination of an ideal match of molecular recognition between the siderophore and the outer membrane receptor/transporter protein to give a selective growth advantage for the producing organism. However, many bacteria do express outer membrane proteins that recognize and then utilize siderophores that are biosynthesized by other bacteria, thus exploiting the biosynthetic efforts of their competitors. As a counter to this iron thievery process, some bacteria synthesize natural siderophore-antibiotic conjugates called sideromycins. There have been efforts to mimic natural sideromycins by design, syntheses, and studies of siderophore-antibiotic conjugates, most of which incorporate well-known antibiotics with the intent of expanding their activity by facilitating active transport of the antibiotic warhead into the targeted bacteria while also circumventing efflux pumps associated with the antibiotic alone (e.g Ji, C. et al., “Exploiting bacterial iron acquisition: siderophore conjugates:, Future Med. Chem. 2012, 4, 297-313).

### SUMMARY OF THE INVENTION

Disclosed herein is a method for reducing the amount of potentially cross-reactive or interfering organisms in an assay designed to detect a detectable indication of viability of a target organism, comprising: obtaining a sample potentially comprising at least one organism that is potentially cross-reactive or interfering in an assay designed to detect a detectable indication of viability of a target organism; contacting the cross-reactive or interfering organism with at least one compound that is involved with the viability of the potentially cross-reactive or interfering organism, wherein the compound is specific to the cross-reactive or interfering organism; and causing the cross-reactive or interfering organism to lose viability without affecting viability of the target organism contacting the sample with a non-replicative transduction particle (NRTP) comprising a reporter nucleic acid molecule encoding a reporter molecule, under conditions such that the NRTP inserts into the target organism the reporter nucleic acid molecule and such that the reporter molecule provides the detectable indication of viability of the target organism.

In some aspects, the at least one compound is a sideromycin. In certain aspects, the sideromycin is a naturally occurring sideromycin. In other aspects, the sideromycin is a synthetic sideromycin. In one aspect, the cross-reactive or interfering organism is Pseudomonas aeruginosa and the compound is a peptidomimetic antimicrobial peptide. In another aspect, the peptide is L27-11. In some aspects, the presence of the detectable indication of viability indicates that the microorganism is viable. In some aspects, the absence of the detectable indication of viability indicates that the microorganism is not viable.

In some aspects, the detectable indication of viability is growth of the microorganism, a marker associated with the microorganism, or a detectable signal associated with the microorganism.

In some aspects, a method disclosed herein further comprises contacting the sample with a reporter nucleic acid molecule encoding a reporter molecule, under conditions such that the reporter molecule enters the microorganism and provides the detectable indication of viability. In some aspects, the reporter system is a non-replicative transduction particle-based reporter system. In some aspects, the at least one microorganism comprises a reporter nucleic acid molecule encoding a reporter molecule.

In some aspects, a method disclosed herein further comprises contacting the sample with a non-replicative transduction particle (NRTP) comprising a reporter nucleic acid molecule encoding a reporter molecule, under conditions such that the NRTP inserts into the microorganism the reporter nucleic acid molecule and such that the reporter molecule provides the detectable indication of viability.

In some aspects, the NRTP is produced from a bacterial cell packaging system that comprises a host bacteria cell, a first nucleic acid construct inside the host bacteria cell, comprising of a bacteriophage genome having a non-functional packaging initiation site sequence, wherein the non-functional packaging initiation site sequence prevents packaging of the bacteriophage genome into the NRTP, and a second nucleic acid construct inside the host bacteria cell and separate from the first nucleic acid construct, comprising of the reporter nucleic acid molecule having a reporter gene and a functional packaging initiation site sequence for facilitating packaging of a replicon of the reporter nucleic acid molecule into the NRTP, wherein the functional second packaging initiation site sequence on the second nucleic acid construct complements the non-functional packaging initiation site sequence in the bacteriophage genome on the first nucleic acid construct.

In some aspects, the reporter nucleic acid molecule is a gene encoding a light-emitting molecule.

In some aspects, the gene is a luciferase gene.

In some aspects, detecting the detectable indication of viability comprises detecting a presence or absence of the reporter molecule. In some aspects, detecting the detectable indication of viability comprises detecting a presence or absence of a reaction mediated by the reporter molecule. In other aspects, detecting the detectable indication of viability comprises detecting a conformation, activity, or other characteristic of the reporter molecule (e.g., fluorescence or ability to bind to or otherwise interact with another molecule).

In some aspects, the microorganism is of the family Enterobacteriaceae, the genus Enterococcus, or the genus Candida. In some aspects, the microorganism is of the genus Escherichia, Mycobacterium, Staphylococcus, Listeria, Clostridium, Streptococcus, Helicobacter, Rickettsia, Haemophilus, Xenorhabdus, Acinetobacter, Bordetella, Pseudomonas, Aeromonas, Actinobacillus, Pasteurella, Vibrio, Legionella, Bacillus, Calotrhrix, Methanococcus, Stenotrophomonas, Chlamydia, Neisseria, Salmonella, Shigella, Campylobacter or Yersinia.

In some aspects, the antimicrobial is a β-lactam or vancomycin. In some aspects, the antimicrobial agent is of the group or class Penicillins, Cephalosporin, Carbapenems, Aminoglycosides, Fluoroquinolone, Lincosamide, Polymyxin, Tetracycline, Macrolide, Oxazolidinone, Streptogramins, Rifamycin, or Glycopeptide. In some aspects, the antimicrobial is Ampicillin, Ampicillin-sulbactam, Pipercillin-tazobactam, Oxacillin, Penicillin, Cefazolin, Cefepime, Cefotaxime, Ceftazidime, Ceftriaxone, Ceftaroline fosomil, Ertapenem, Imipenem, Meropenem, Amikacin, Gentamicin, Gentamicin Synergy, Streptomycin Synergy, Tobramycin, Ciprofloxacin, Levofloxacin, Clindamycin, Colistin, Daptomycin, Doxycycline, Erythromycin, Linezolid, Nitrofurantoin, Quinupristin-dalfopristin, Rifampin, Tigecycline, Trimethoprim-sulfamethoxazole, fosfomycin, cefoxitin, tetracycline, moxifloxacin, or tedizolid.

In some aspects, detecting the detectable indication of viability comprises observing the growth of the microorganism, optionally wherein growth is observed using cell culture.

In some aspects, the sample is contacted with the antimicrobial agent prior to contacting the sample with the compound. In some aspects, the sample is contacted with the compound prior to contacting the sample with the antimicrobial agent, or wherein the sample is contacted with the compound and the agent simultaneously. In some aspects, the sample, compound, and a reporter nucleic acid are contacted with each other in any sequential permutation or simultaneously.

Also disclosed herein is a method of classifying a microorganism as Enterobacteriaceae in origin or non-Enterobacteriacea in origin, comprising obtaining a sample containing said microorganism; contacting said sample with a sideromycin combination comprising Albomycin and SalmycinA; contacting said sample with a non-replicative transduction particle (NRTP) comprising a reporter nucleic acid molecule encoding a reporter molecule, under conditions such that the NRTP inserts into the microorganism the reporter nucleic acid molecule and such that the reporter molecule provides a detectable indication of viability of the microorganism; wherein said microorganism is classified as Enterobacteriaceae in origin if the detectable indication of viability of the microorganism is reduced by greater than 50% from the presence of the sideromycin combination, and wherein said microorganism is classified as non-Enterobacteriaceae in origin if the detectable indication of viability of the microorganism is reduced by less than 50% from the presence of the sideromycin combination.

In some aspects, the reporter molecule is a light emitting molecule and the detectable indication of viability of the microorganism is a light signal. In other aspects, the light emitting molecule is a luciferase molecule.

In some aspects, the sideromycin combination comprises Albomycin at a concentration ranging from 3 μg/mL to 10 μg/mL and SalmycinA at a concentration ranging from 0.05 μg/mL to 0.25 μg/mL.

Also disclosed herein is a kit for reducing the amount of potentially cross-reactive or interfering organisms in an assay designed to detect a target organism comprising a compound that causes the cross-reactive or interfering organism to lose viability without affecting viability of the target organism and a NRTP comprising a reporter nucleic acid molecule encoding a reporter molecule, under conditions such that the NRTP inserts into the target organism the reporter nucleic acid molecule and such that the reporter molecule provides the detectable indication of viability of the target organism.

In some aspects, the NRTP is produced from a bacterial cell packaging system that comprises a host bacteria cell, a first nucleic acid construct inside the host bacteria cell, comprising of a bacteriophage genome having a non-functional packaging initiation site sequence, wherein the non-functional packaging initiation site sequence prevents packaging of the bacteriophage genome into the NRTP, and a second nucleic acid construct inside the host bacteria cell and separate from the first nucleic acid construct, comprising of the reporter nucleic acid molecule having a reporter gene and a functional packaging initiation site sequence for facilitating packaging of a replicon of the reporter nucleic acid molecule into the NRTP, wherein the functional second packaging initiation site sequence on the second nucleic acid construct complements the non-functional packaging initiation site sequence in the bacteriophage genome on the first nucleic acid construct.

In some aspects, the compound is a sideromycin combination comprising Albomycin and SalmycinA. In some aspects, the sideromycin combination comprises Albomycin at a concentration ranging from 3 μg/mL to 10 μg/mL and SalmycinA at a concentration ranging from 0.05 μg/mL to 0.25 μg/mL.

In some aspects, the reporter nucleic acid molecule encodes a luciferase gene and the reporter molecule is a luciferase molecule.

### BRIEF DESCRIPTION OF THE DRAWINGS

These and other features, aspects, and advantages of the present disclosure will become better understood with regard to the following description, and accompanying drawings, where:

FIG. 1 is a graphical representation of the Smarticles assay with and without sideromycins

FIG. 2 shows the Relative Luminometer Unit (RLU) response in the presence of Albomycin (6 μg/mL) and SalmycinA (0.128 μg/mL) across various bacterial species in the experiment of Example 1

FIG. 3 shows the RLU kinetics in Escherichia coli.

FIG. 4 shows the RLU kinetics in Acinetobacter baumanii.

FIG. 5 shows organism classification based on the Relative Luminometer Unit (RLU) response in the presence of Albomycin (6 μg/mL) and SalmycinA (0.128 μg/mL).

FIG. 6 shows graphical distribution of isolate species based on the Relative Luminometer (RLU) response in the presence of Albomycin (6 μg/mL) and SalmycinA (0.128 μg/mL).

### DETAILED DESCRIPTION OF THE INVENTION

Terms used in the claims and specification are defined as set forth below unless otherwise specified.

“Siderophores” are small, high-affinity iron-chelating compounds secreted by microorganisms such as bacteria and fungi and serving to transport iron across cell membranes.

“Sideromycins” are a group of antibiotics linked to siderophores by covalent bonds. Sideromycins can actively bypass permeability barriers (membranes) to deliver the drug inside the target bacterial cell, irrespective of the size and polarity of the antibiotic moiety contained into it. Examples of naturally occurring sideromycins are albomycin and salmycin which are described in Braun et al., Biometals 2009, 22:3-13 and incorporated herein by reference in its entirety. Examples of synthetic sideromycins include cefiderocol, as described in Ito et al., Antimicrob Agents Chemother. 2017, 62(1): e01454-17; biomimetic siderophore-aminopenicillin compounds described in Mollmann et al., Biometals 2009, 22:615-624; enterobactin-ampicillin/amoxicillin as described in Zheng et al., J. Am. Chem. Soc. 2014, 136: 9677-9691; pyoverdin-ampicillin as described in Kinzel et al., J. Antibiotics 1998, 51(5): 499-507; pyochelin-norfloxacin as described in Rivault et al., Bioorg Med Chem Lett. 2007 Feb. 1, 17(3): 640-644; siderophore-monocarbam as described in Flanagan et al, ACS Med. Chem. Lett. 2011, 2: 385-390; and synthetic siderophore-daptomycin as described in Ghosh et al., J. Med. Chem. 2017, 60: 4577-4583, whose disclosures are all incorporated herein by reference in their entireties.

As used herein, “reporter nucleic acid molecule” refers to a nucleotide sequence comprising a DNA or RNA molecule. The reporter nucleic acid molecule can be naturally occurring or an artificial or synthetic molecule. In some embodiments, the reporter nucleic acid molecule is exogenous to a host cell and can be introduced into a host cell as part of an exogenous nucleic acid molecule, such as a plasmid or vector. In other embodiments, the reporter nucleic acid molecule comprises a reporter gene encoding a reporter molecule (e.g., reporter enzyme, protein). In some embodiments, the reporter nucleic acid molecule is referred to as a “reporter construct” or “nucleic acid reporter construct.”

A “reporter molecule” or “reporter” refers to a molecule (e.g., nucleic acid-derived or amino acid-derived) that confers onto an organism a detectable or selectable phenotype. The detectable phenotype can be colorimetric, fluorescent or luminescent, for example. Reporter molecules can be expressed from reporter genes encoding enzymes mediating luminescence reactions (luxA, luxB, luxAB, luc, ruc, nluc), genes encoding enzymes mediating colorimetric reactions (lacZ, HRP), genes encoding fluorescent proteins (GFP, eGFP, YFP, RFP, CFP, BFP, mCherry, near-infrared fluorescent proteins), nucleic acid molecules encoding affinity peptides (His-tag, 3X-FLAG), and genes encoding selectable markers (ampC, tet(M), CAT, erm). The reporter molecule can be used as a marker for successful uptake of a nucleic acid molecule or exogenous sequence (plasmid) into a cell. The reporter molecule can also be used to indicate the presence of a target gene, target nucleic acid molecule, target intracellular molecule, or a cell. The reporter molecule can also be used to indicate the viability of a cell. Alternatively, the reporter molecule can be a nucleic acid, such as an aptamer or ribozyme.

In some aspects, the reporter nucleic acid molecule is operatively linked to a promoter. In other aspects, the promoter can be chosen or designed to contribute to the reactivity and cross-reactivity of the reporter system based on the activity of the promoter in specific cells (e.g., specific species) and not in others. In certain aspects, the reporter nucleic acid molecule comprises an origin of replication. In other aspects, the choice of origin of replication can similarly contribute to reactivity and cross-reactivity of the reporter system, when replication of the reporter nucleic acid molecule within the target cell contributes to or is required for reporter signal production based on the activity of the origin of replication in specific cells (e.g., specific species) and not in others. In some embodiments, the reporter nucleic acid molecule forms a replicon capable of being packaged (e.g., as concatameric DNA) into a progeny virus during virus replication. In other aspects, the reporter nucleic acid molecule includes factors that influence the transcription or translation of the reporter gene (e.g., specific ribosome binding sites, codon usage) that can similarly contribute to reactivity and cross-reactivity of the reporter system.

As used herein, the term “transcript” refers to a length of nucleotide sequence (DNA or RNA) transcribed from a DNA or RNA template sequence or gene. The transcript can be a cDNA sequence transcribed from an RNA template or an mRNA sequence transcribed from a DNA template. The transcript can be protein coding or non-coding. The transcript can also be transcribed from an engineered nucleic acid construct.

As used herein, a “target transcript” refers to a portion of a nucleotide sequence of a DNA sequence or an mRNA that is naturally formed by a target cell including that formed during the transcription of a target gene and mRNA that is a product of RNA processing of a primary transcription product. The target transcript can also be referred to as a cellular transcript or naturally occurring transcript.

“Introducing into a cell,” when referring to a nucleic acid molecule or exogenous sequence (e.g., plasmid, vector, construct), means facilitating uptake or absorption into the cell, as is understood by those skilled in the art. Absorption or uptake of nucleic acid constructs or transcripts can occur through unaided diffusive or active cellular processes, or by auxiliary agents or devices including via the use of bacteriophage, virus, transduction particles, liposomes, polymers, virus-like particles, and ballistic means. The meaning of this term is not limited to cells in vitro; a nucleic acid molecule may also be “introduced into a cell,” wherein the cell is part of a living organism. In such instance, introduction into the cell will include the delivery to the organism. For example, for in vivo delivery, nucleic acid molecules, constructs or vectors can be injected into a tissue site or administered systemically. In vitro introduction into a cell includes methods known in the art, such as transformation, electroporation, transduction, and lipofection. Further approaches are described herein or known in the art.

A “mechanism for the antimicrobial susceptibility phenotype” refers to one or more mechanisms (e.g., one or more genes, mRNAs, and/or proteins) that are involved in imparting resistance or susceptibility of an organism to an antimicrobial agent.

As used herein, the term “molecule” means any compound, including, but not limited to, a small molecule, peptide, protein, sugar, nucleotide, nucleic acid, lipid, etc., and such a compound can be natural or synthetic.

An “antimicrobial agent” refers to a compound that can kill, inhibit the growth, or otherwise compromise the viability of one or more microorganisms. Antimicrobial agents include antibiotics, antifungals, antiprotozoals, antivirals, and other compounds.

A “detectable indication of viability” refers to an indicator associated with a cell that can be observed and that demonstrates whether the cell is more or less viable or if its viability is affected, e.g., relative to a control cell, where the control cell can be the same cell at a different time point or a separate cell. Examples include one or more signals, one or more reporters, one or more markers, growth or lack thereof, light (e.g., light emitted by a luciferase) or lack thereof, etc.

A virus-based reporter or bacteriophage-based reporter can refer to a virus or bacteriophage, respectively, which has been modified such that a reporter gene has been inserted in its genome.

A “transduction particle” refers to a virus capable of delivering a non-viral nucleic acid molecule into a cell. The virus can be a bacteriophage, adenovirus, etc. A transduction particle reporter can be synonymous with a virus or bacteriophage-based reporter.

A “non-replicative transduction particle” (NRTP) refers to a virus capable of delivering a non-viral nucleic acid molecule into a cell, but does not package its own replicated viral genome into the transduction particle. The virus can be a bacteriophage, adenovirus, etc. NRTPs and methods of making the same are described in detail in U.S. Pat. No. 9,388,453, which is incorporated by reference in its entirety for all purposes.

A “plasmid” is a small DNA molecule that is physically separate from, and can replicate independently of, chromosomal DNA within a cell. Most commonly found as small circular, double-stranded DNA molecules in bacteria, plasmids are sometimes present in archaea and eukaryotic organisms. Plasmids are considered replicons, capable of replicating autonomously within a suitable host.

A “vector” is a molecule that includes nucleic acids that can be used as a vehicle to carry genetic material into a cell, where it can be integrated, replicated and/or expressed.

A “virus” is a small infectious agent that replicates only inside the living cells of other organisms. Virus particles (known as virions) include two or three parts: i) the genetic material made from either DNA or RNA molecules that carry genetic information; ii) a protein coat that protects this nucleic acid; and in some cases, iii) an envelope of lipids that surrounds the protein coat. When referring to a virus that infects bacteria, the terms “virus”, “phage” and “bacteriophage” are used interchangeably in the specification.

“Specific binding” refers to the ability of two molecules to bind to each other in preference to binding to other molecules in the environment. Typically, “specific binding” discriminates over adventitious binding in a reaction by at least two-fold, more typically by at least 10-fold, often at least 100-fold or greater. Typically, the affinity or avidity of a specific binding reaction, as quantified by a dissociation constant, is about 10⁻⁷ M or stronger (e.g., about 10⁻⁸ M, 10⁻⁹ M or even stronger).

The term “ameliorating” refers to any therapeutically beneficial result in the treatment of a disease state, e.g., a disease state, including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “mammal” as used herein includes both humans and non-humans and include but is not limited to humans, non-human primates, canines, felines, murines, bovines, equines, and porcines.

The term “microorganism” means prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

The terms “marker” or “markers” encompass, without limitation, lipids, lipoproteins, proteins, cytokines, chemokines, growth factors, peptides, nucleic acids, genes, and oligonucleotides, together with their related complexes, metabolites, mutations, variants, polymorphisms, modifications, fragments, subunits, degradation products, elements, and other analytes or sample-derived measures. A marker can also include mutated proteins, mutated nucleic acids, variations in copy numbers, and/or transcript variants.

The term “sample” can include a single cell or multiple cells or fragments of cells or an aliquot of body fluid, taken from an environment or subject, by means including venipuncture, excretion, ejaculation, massage, biopsy, needle aspirate, lavage sample, scraping, surgical incision, swabbing, or intervention or other means known in the art. Typically, the sample provided in the methods disclosed herein is an in vitro sample.

The term “subject” encompasses a cell, tissue, or organism, human or non-human, whether in vivo, ex vivo, or in vitro, male or female.

“G,” “C,” “A” and “U” each generally stand for a nucleotide that contains guanine, cytosine, adenine, and uracil as a base, respectively. “T” and “dT” are used interchangeably herein and refer to a deoxyribonucleotide wherein the nucleobase is thymine, e.g., deoxyribothymine. However, it will be understood that the term “ribonucleotide” or “nucleotide” or “deoxyribonucleotide” can also refer to a modified nucleotide, as further detailed below, or a surrogate replacement moiety. The skilled person is well aware that guanine, cytosine, adenine, and uracil may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine can be replaced in the nucleotide sequences by a nucleotide containing, for example, inosine. Sequences comprising such replacement moieties are embodiments.

As used herein, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Complementary sequences are also described as binding to each other and characterized by binding affinities.

The term “sufficient amount” means an amount sufficient to produce a desired effect, e.g., an amount sufficient to produce a detectable signal from a cell.

The term “therapeutically effective amount” is an amount that is effective to ameliorate a symptom of a disease. A therapeutically effective amount can be a “prophylactically effective amount” as prophylaxis can be considered therapy.

It must be noted that, as used in the specification and the appended claims, the singular forms “a,” “an” and “the” include plural referents unless the context clearly dictates otherwise.

### NRTPs and Reporter Assays

Non-replicative transduction particles (NRTPs) and methods of producing NRTPs are described in U.S. Pat. No. 9,388,453, and in U.S. Patent Application Publication No. 2017/0166907 (the entire disclosures of both are incorporated by reference in their entireties for all purposes. In some embodiments, NRTPs are produced in a bacterial cell packaging system using Disruption/Complementation-based methods. This non-replicative transduction particle packaging system is based on introducing a mutation, silent mutation, insertion, or a deletion into a component of the genome of a virus/bacteriophage that is recognized by the viral/phage packaging machinery as the element from which genomic packaging is initiated during viral/phage production. Examples of such an element include the pac-site sequence of pac-type bacteriophages and the cos-site sequence of cos-type bacteriophages.

Because these packaging initiation sites are often found within coding regions of genes that are essential to virus/bacteriophage production, the mutation, silent mutation, insertion, or a deletion is introduced such that the pac-site is no longer recognized as a site of packaging initiation by the viral/phage packaging machinery. At the same time, in the case of a silent mutation, the mutation does not disrupt the gene in which the site is encoded. By rendering the packaging site sequence non-functional, the mutated virus/bacteriophage is able to undergo a lytic cycle, but is unable to package its genomic DNA into its packaging unit.

An exogenous reporter nucleic acid molecule, such as plasmid DNA, can be introduced into a host bacteria cell that has been lysogenized with a viral/phage genome with a non-functional packaging initiation site sequence. The exogenous reporter nucleic acid molecule can include a native functional packaging initiation site sequence and, in the case where the gene encoding the packaging initiation site sequence is disrupted, the exogenous reporter nucleic acid molecule also includes a corresponding native functional gene. The exogenous reporter nucleic acid molecule can be introduced into the host bacteria cell and replicated in the cell. When the mutated virus/bacteriophage is undergoing a lytic cycle, the expressed viral/phage packaging machinery packages the exogenous reporter nucleic acid molecule with the functional packaging initiation site sequence into the viral packaging unit. The viral/phage genome is not packaged into the packaging unit because its packaging initiation site sequence has been disrupted.

Therefore, the present invention contemplates the use of a bacterial cell packaging system for packaging a reporter nucleic acid molecule into a NRTP for introduction into a cell, which comprises a host bacteria cell, a first nucleic acid construct inside the host bacteria cell, comprising of a bacteriophage genome having a non-functional packaging initiation site sequence, wherein the non-functional packaging initiation site sequence prevents packaging of the bacteriophage genome into the NRTP, and a second nucleic acid construct inside the host bacteria cell and separate from the first nucleic acid construct, comprising of the reporter nucleic acid molecule having a reporter gene and a functional packaging initiation site sequence for facilitating packaging of a replicon of the reporter nucleic acid molecule into the NRTP, wherein the functional second packaging initiation site sequence on the second nucleic acid construct complements the non-functional packaging initiation site sequence in the bacteriophage genome on the first nucleic acid construct.

In some embodiments, constructs (including NRTPs) comprise a reporter nucleic acid molecule including a reporter gene. The reporter gene can encode a reporter molecule, and the reporter molecule can be a detectable or selectable marker. In certain embodiments, the reporter gene encodes a reporter molecule that produces a detectable signal when expressed in a cell.

In certain embodiments, the reporter molecule can be a fluorescent reporter molecule, such as, but not limited to, a green fluorescent protein (GFP), enhanced GFP, yellow fluorescent protein

(YFP), cyan fluorescent protein (CFP), blue fluorescent protein (BFP), red fluorescent protein (RFP) or mCherry, as well as near-infrared fluorescent proteins. In other embodiments, the reporter molecule can be an enzyme mediating luminescence reactions (luxA, luxB, luxAB, luc, ruc, nluc, etc.). Reporter molecules can include a bacterial luciferase, a eukaryotic luciferase, an enzyme suitable for colorimetric detection (lacZ, HRP), a protein suitable for immunodetection, such as affinity peptides (His-tag, 3X-FLAG), a nucleic acid that function as an aptamer or that exhibits enzymatic activity (ribozyme), or a selectable marker, such as an antibiotic resistance gene (ampC, tet(M), CAT, erm). Other reporter molecules known in the art can be used for producing signals to detect target nucleic acids or cells.

In other aspects, the reporter molecule comprises a nucleic acid molecule. In some aspects, the reporter molecule is an aptamer with specific binding activity or that exhibits enzymatic activity (e.g., aptazyme, DNAzyme, ribozyme).

Delivery of cell reporter nucleic acid molecules may be accomplished by various means including electroporation, chemical, biolistic, and glass bead transformation, transduction, transfection, vectors, conjugation, including, but not limited to, delivery via nucleic acid delivery vehicles including bacteriophage, virus, spheroplast, liposomes, virus-like particles, lipid-DNA complexes, lipoplexes, polymer-DNA complexes, polyplexes, etc.

The present invention relates to the use of sideromycins, i.e. siderophores covalently linked to an antimicrobial agent (e.g. an antibiotic) as additives in non-replicative transduction particle reporter-based assays to either limit cross-reactivity of unwanted organisms or to identify the organism being run on an antibiotic susceptibility (AST) assay. Addition of sideromycins removes or reduces the production of signals (e.g. light from a luciferase assay) from bacteria that are sensitive to them, allowing for prevention of cross-reactivity in cell reporter assays and/or family, genus, and potentially species level identification when performing AST testing. This technology is especially useful in assays where it is difficult to control cross-reactivity for certain unwanted bacterial strains and species. The sensitivity, specificity and species-level conservative of active iron transporter systems (i.e. siderophores) means this technique could be rapidly and universally applicable to cell reporter assays using non-replicative transduction particles (NRTP) such as the Smarticles system.

Sidermomycins have been considered as an antibiotic therapeutic for both Gram-positive and Gram-negative bacterial infections (see Braun, V. et al., “Sideromycins: tools and antibiotics”, Biometals (2009) 22:3-13), but they have not been used in diagnostic assays. The high specificity and low concentrations needed for effective use make them ideal for cell reporter assays using NRTPs. The limited spectrum of sideromycin activity has hindered their use in therapeutics. However, this limitation is beneficial for bacterial identification assays that utilize cell reporter NRTPs. Additionally, concerns over developed resistance to sideromycins when used as an antimicrobial are not an issue in a NRTP-based diagnostic assay. As a small molecule, sideromycins are easily incorporated in any assay format. Furthermore, the ability to tune sideromycin specificity by using different siderophores or different antibiotic moieties provides a powerful tool to achieve assay specificity with NRTPs.

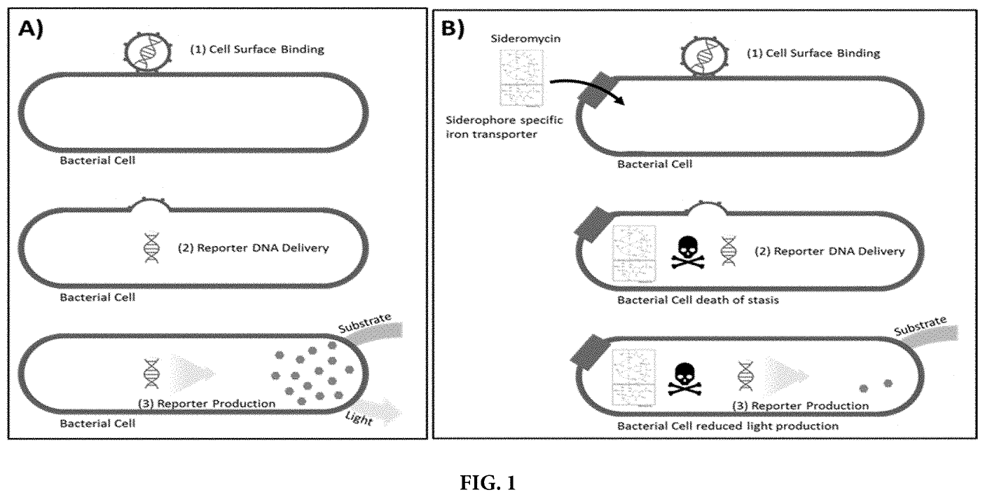

A schematic on how sideromycins would function in a NRTP-based reporter assay (also referred as Smarticles assay) is shown in FIG. 1. Panel A) shows the function of Smarticles in the absence of sideromycins. Smarticles are able to transduce a permissive host and the metabolic activity of the host cell allows it to produce the reporter protein (luciferase enzyme) and subsequently produce light in the presence of substrate. Panel B) shows the Smarticles assay in the presence of sideromycins. Sideromycins are imported into the bacteria with a known siderophore specific iron transport system, eliciting either bacteriocidal or bacteriostatic action and preventing light production by the bacteria. While the Smarticles are still able to transduce such bacterial cells, due to the antibiotic that is conjugated to the siderophore, the host metabolism is not able to produce the reporter protein. Light is either not produced or produced at greatly decreased level after the addition of substrate.

Therefore, the present invention contemplates methods of performing a NRTP-based reporter assay for detection of a microorganism (bacteria) in a sample. In this assay, the sideromycin is introduced to a sample that contains both wanted and unwanted bacteria. A predetermined amount of time allows the sideromycins to be imported into the unwanted bacteria via the corresponding active transport system. The antibiotic conjugated within the sideromycin will then affect the metabolism and/or viability of the unwanted bacteria to reduce or prevent the production of the reporter protein such that the production of light (due to expression of the reporter protein) is reduced in the sideromycin-sensitive cells. On the other hand, bacteria cells that lack the corresponding active transport proteins or where the antibiotics are ineffective will not have any reduction in light production and will therefore be detectable. Therefore, the response to the presence of one or more sideromycins allows for the detection of specific families, genera or species of microorganisms in the NRTP-based reporter assay, which allows for their identification.

### EXAMPLES

### Example 1: Specific Reduction of Light Signal by Albomycin/Salmycin Combination

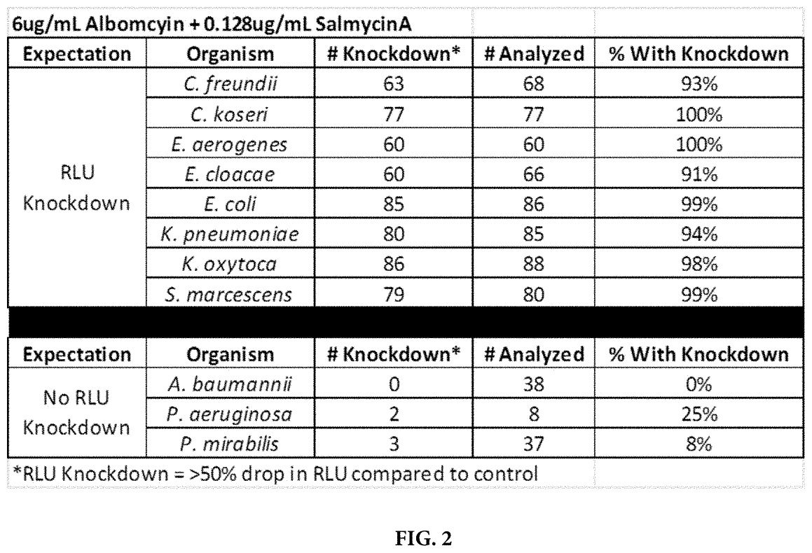

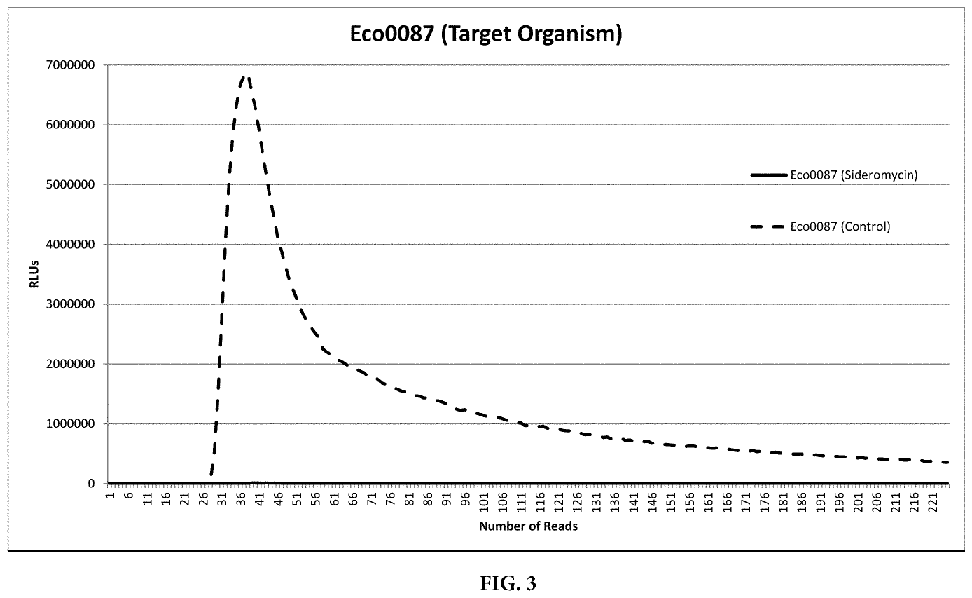

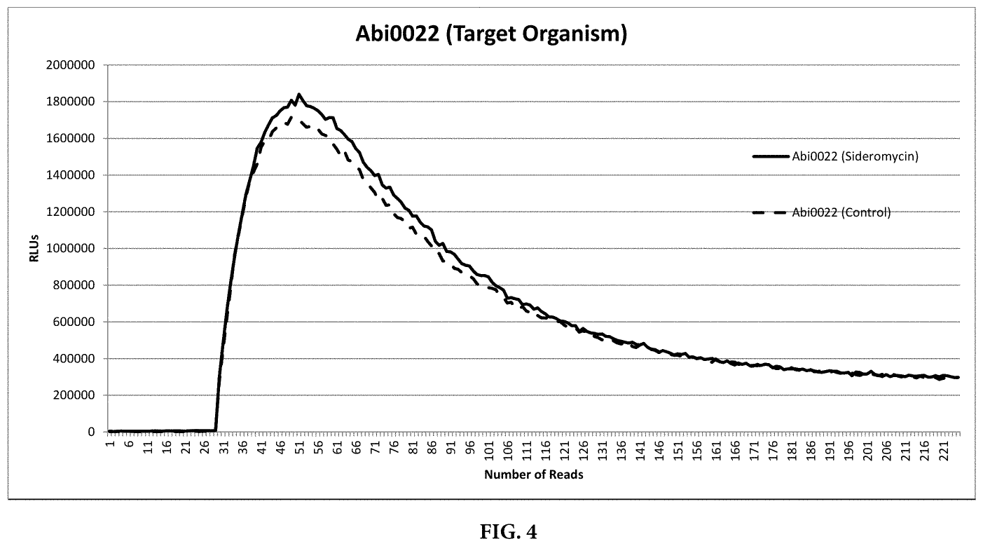

In this example, an Enterobacteriaceae reporter system was used in conjunction with Albomycin at a concentration of 6 μg/mL and SalmycinA at a concentration of 0.128 μg/mL across eight Enterobacteriaceae species, (C. freundii, C. koseri, E. aerogenes, E. cloacae, E. coli, K pneumonia, K. oxytoca, S. marcenscens) and three non-Enterobacteriaceae species (A. baumannii, P. aeruginosa, P. mirabilis) in which light had been detected in the absence of the sideromycins. The assay involves an initial 2.5 hour pre-treatment of bacterial cells at a concentration of 5.0 E+05 CFU/mL with the Albomycin/SalmycinA Combination in assay media (10 g/L Tryptone+5 g/L Yeast Extract+5% PEG8000). Following the pre-treatment step, both non-replicative transduction particles (NRTPs) and transduction salts (1M MgCl₂+0.5M CaC₂) were added to the reaction and incubated for 2 hours—this allows for transduction of the reporter molecule within the NRTPs that contained the luciferase gene, luxAB. To gauge the level of Relative Luminometer Unit (RLU) reduction or knockdown achieved, light production from untreated bacteria (control) were compared to light production from bacteria treated with the sideromycin combination and the results of the experiment are shown in FIG. 2. For all eight Enterobacteriaceae species tested, over 90% of all isolates showed reduction or knockdown in light production as measured by RLU by greater than 50%. In contrast, the three non-Enterobacteriaceae species, which should be unaffected by the sideromycin combination, had fewer than 25% of isolates showing no reduction in light production. The kinetics of light production from this experiment are shown in two example strains: Escherichia coli (Eco0087, FIG. 3) and Acinetobacter baumannii (Abi0022 FIG. 4).

### Example 2: Use of Sideromycins to Improve Bacterial Identification

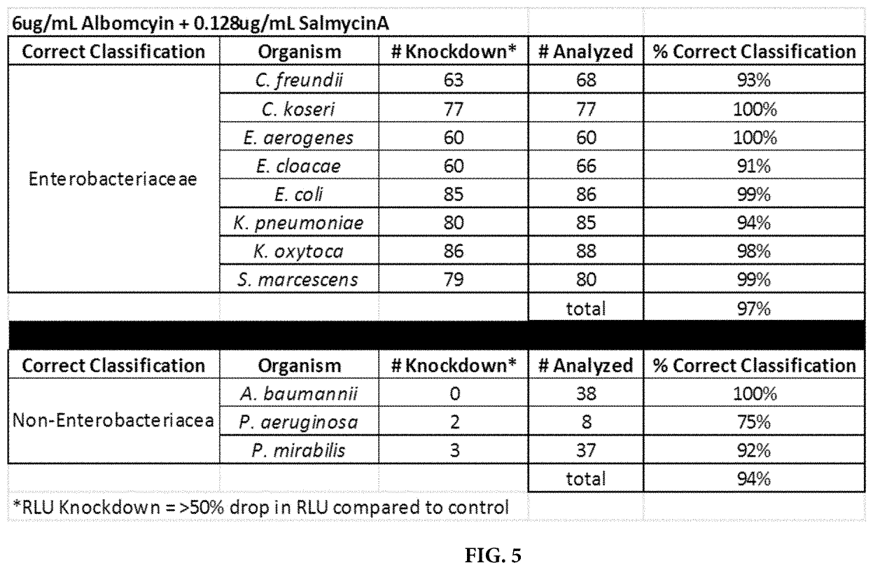

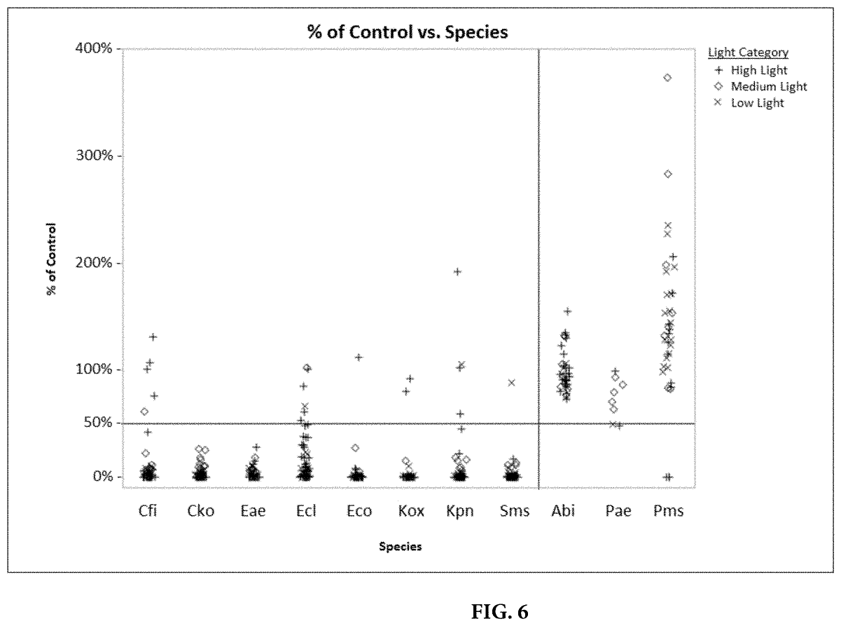

Utilizing the same experiment and data set from Example 1, additional analyses were performed to determine the ability of sideromycins to classify the source of light production as either Enterobacteriaceae or non-Enterobacteriaceae in origin. Enterobacteriaceae organisms with light production are expected to exhibit RLU knockdown/reduction in the presence of the sideromycin combination of Albomycin and SalmycinA. Conversely, non-Enterobacteriaceae organisms with light production are not expected to exhibit RLU knockdown/reduction in the presence of the sideromycin combination. Results from the additional analyses are shown in FIG. 5, where as a whole, 97% of light production (due to Enterobacteriaceae) was correctly classified, and 94% of light production (due to non-Enterobacteriaceae) was correctly classified. This is further illustrated in FIG. 6, where the Enterobacteriaceae and non-Enterobacteriaceae organisms can be separated into distinct groups based on their response to the Albomycin and SalmycinA combination.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference, in their entirety, for all purposes.

1. A method for reducing the amount of potentially cross-reactive or interfering organisms in an assay designed to detect a detectable indication of viability of a target organism, comprising:
obtaining a sample potentially comprising at least one organism that is potentially cross-reactive or interfering in an assay designed to detect the detectable indication of viability of the target organism;
contacting the cross-reactive or interfering organism with at least one compound that is involved with the viability of the potentially cross-reactive or interfering organism, wherein the compound is specific to the cross-reactive or interfering organism and causing the cross-reactive or interfering organism to lose viability without affecting viability of the target organism;
contacting the sample with a non-replicative transduction particle (NRTP) comprising a reporter nucleic acid molecule encoding a reporter molecule, under conditions such that the NRTP inserts into the target organism the reporter nucleic acid molecule and such that the reporter molecule provides the detectable indication of viability of the target organism.

2. The method of claim 1, wherein the at least one compound is a sideromycin.

3. The method of claim 2, wherein the sideromycin is a naturally occurring sideromycin.

4. The method of claim 2, wherein the sideromycin is a synthetic sideromycin.

5. The method of claim 1, wherein the NRTP is produced from a bacterial cell packaging system that comprises a host bacteria cell, a first nucleic acid construct inside the host bacteria cell, comprising of a bacteriophage genome having a non-functional packaging initiation site sequence, wherein the non-functional packaging initiation site sequence prevents packaging of the bacteriophage genome into the NRTP, and a second nucleic acid construct inside the host bacteria cell and separate from the first nucleic acid construct, comprising of the reporter nucleic acid molecule having a reporter gene and a functional packaging initiation site sequence for facilitating packaging of a replicon of the reporter nucleic acid molecule into the NRTP, wherein the functional second packaging initiation site sequence on the second nucleic acid construct complements the non-functional packaging initiation site sequence in the bacteriophage genome on the first nucleic acid construct.

6. The method of claim 5, wherein the reporter gene is a luciferase gene.

7. The method of claim 1, wherein the target organism is of the family Enterobacteriacaeae.

8. A method of classifying a microorganism as Enterobacteriaceae in origin or non-Enterobacteriacea in origin, comprising:
obtaining a sample containing said microorganism;
contacting said sample with a sideromycin combination comprising Albomycin and SalmycinA;
contacting said sample with a non-replicative transduction particle (NRTP) comprising a reporter nucleic acid molecule encoding a reporter molecule, under conditions such that the NRTP inserts into the microorganism the reporter nucleic acid molecule and such that the reporter molecule provides a detectable indication of viability of the microorganism;
wherein said microorganism is classified as Enterobacteriaceae in origin if the detectable indication of viability of the microorganism is reduced by greater than 50% from the presence of the sideromycin combination, and wherein said microorganism is classified as non-Enterobacteriaceae in origin if the detectable indication of viability of the microorganism is reduced by less than 50% from the presence of the sideromycin combination.

9. The method of claim 8, wherein the reporter molecule is a light emitting molecule and the detectable indication of viability of the microorganism is a light signal.

10. The method of claim 9, wherein the light emitting molecule is a luciferase molecule.

11. The method of claim 8, wherein the sideromycin combination comprises Albomycin at a concentration ranging from 3 μg/mL to 10 μg/mL and SalmycinA at a concentration ranging from 0.05 μg/mL to 0.25 μg/mL.

12. A kit for reducing the amount of potentially cross-reactive or interfering organisms in an assay designed to detect a target organism comprising:
a compound that causes the cross-reactive or interfering organism to lose viability without affecting viability of the target organism; and
a non-replicative transduction particle (NRTP) comprising a reporter nucleic acid molecule encoding a reporter molecule, under conditions such that the NRTP inserts into the target organism the reporter nucleic acid molecule and such that the reporter molecule provides a detectable indication of viability of the target organism.

13. The kit of claim 12, wherein the NRTP is produced from a bacterial cell packaging system that comprises a host bacteria cell, a first nucleic acid construct inside the host bacteria cell, comprising of a bacteriophage genome having a non-functional packaging initiation site sequence, wherein the non-functional packaging initiation site sequence prevents packaging of the bacteriophage genome into the NRTP, and a second nucleic acid construct inside the host bacteria cell and separate from the first nucleic acid construct, comprising of the reporter nucleic acid molecule having a reporter gene and a functional packaging initiation site sequence for facilitating packaging of a replicon of the reporter nucleic acid molecule into the NRTP, wherein the functional second packaging initiation site sequence on the second nucleic acid construct complements the non-functional packaging initiation site sequence in the bacteriophage genome on the first nucleic acid construct.

14. The kit of claim 13, wherein the reporter gene is a luciferase gene.

15. The kit of claim 12, wherein the compound is a sideromycin combination comprising Albomycin and SalmycinA.
